(19) United States
(12) Patent Application Publication
(10) Pub. No.: US 2007/0197615 A1
(43) Pub. Date: Aug. 23, 2007

(54) Process for the preparation of the PPAR alpha agonist NS-220

(76) Inventors: Thomas Hartung, Loerrach (DE); Christian Jenny, Basel (CH); Francois Montavon, Delemont (CH); Pius Waldmeier, Wegenstetten (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/706,768

(22) Filed: Feb. 15, 2007

(30) Foreign Application Priority Data
Feb. 21, 2006 (EP) 06110192.9

(51) Int. Cl. A61K 31/422; C07D 413/02

(52) U.S. Cl. 514/374; 548/238

(57) ABSTRACT

The present invention is concerned with a novel process for the preparation of compounds of formula (I)

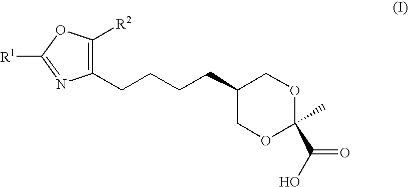

wherein R¹ and R² are as defined in the description and claims. The compounds of formula (I) are pharmaceutically active substances.

This application claims the benefit of European Application No. 06110192.9, filed Feb. 21, 2006, which is hereby incorporated by reference in its entirety.

### FIELD OF THE INVENTION

The present invention is directed to a novel process for the preparation of dioxane derivatives, especially with a process for the preparation of compounds of formula (I):

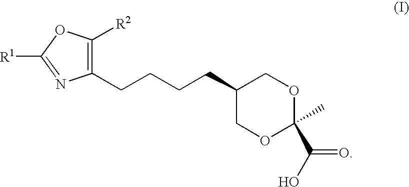

All documents cited or relied upon below are expressly incorporated herein by reference.

### BACKGROUND

The compounds of formula (I), such as, for example, [cis]-2-Methyl-5-[4-(5-methyl-2-p-tolyl-oxazol-4-yl)-butyl]-[1,3]dioxane-2-carboxylic acid, are pharmaceutically active compounds. These compounds, such as, for example, [cis]-2-Methyl-5-[4-(5-methyl-2-p-tolyl-oxazol-4-yl)-butyl]-[1,3]dioxane-2-carboxylic acid, are known in the art and are described for example in International Patent Application WO 01/90087. They are especially useful for the prophylaxis and/or treatment of metabolic diseases, e.g. dyslipidemia.

Methods for the preparation of compounds of formula (I), such as, for example, [cis]-2-Methyl-5-[4-(5-methyl-2-p-tolyl-oxazol-4-yl)-butyl]-[1,3]dioxane-2-carboxylic acid have been described in WO 01/90087. However, these methods include a large number of individual reaction steps.

Further, the methods known in the art exhibit a low yield or other disadvantages, which makes them unsuitable for the commercial large scale production.

### SUMMARY OF THE INVENTION

In an embodiment of the present invention, provided is a process for the preparation of a compound of formula (I)

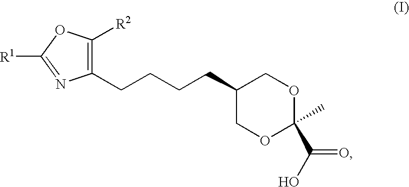

comprising the steps of reacting a compound of formula (II)

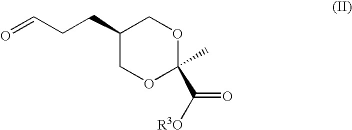

with a compound of formula (III)

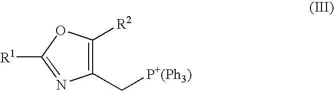

to obtain a compound of formula (IV)

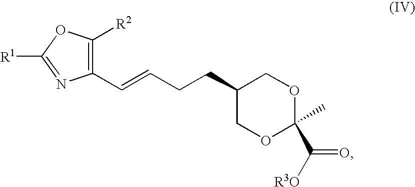

and hydrogenating and saponifying the compound of formula (IV) to obtain the compound of formula (I),
wherein
R¹ is phenyl which is optionally substituted with 1-3 substituents independently selected from the group consisting of halogen, lower-alkyl, lower-alkoxy, fluoro-lower-alkyl and fluoro-lower-alkoxy,
R² is hydrogen or lower-alkyl, and
R³ is hydrogen, lower-alkyl, cycloalkyl or fluoro-lower-alkyl.

In another embodiment of the present invention, provided is a compound selected from the group consisting of:
[cis]-5-But-3-enyl-2-methyl-[1,3]dioxane-2-carboxylic acid ethyl ester,
[cis]-2-Methyl-5-(3-oxo-propyl)-[1,3]dioxane-2-carboxylic acid ethyl ester,
(5-Methyl-2-p-tolyl-oxazol-4-ylmethyl)-triphenyl-phosphonium chloride,
[cis]-2-Methyl-5-[4-(5-methyl-2-p-tolyl-oxazol-4-yl)-but-3-enyl]-[1,3]acid ethyl ester,
[cis]-5-(3-Benzyloxy-propyl)-2-methyl-[1,3]dioxane-2-carboxylic acid ethyl ester,
[cis]-5-(3-Hydroxy-propyl)-2-methyl-[1,3]dioxane-2-carboxylic acid ethyl ester, and
2-[3-(4-Methoxy-benzyloxy)-propyl]-propane-1,3-diol.

### DETAILED DESCRIPTION

It has been found that using the processes according to the present invention, compounds of formula (I) and particularly [cis]-2-Methyl-5-[4-(5-methyl-2-p-tolyl-oxazol-4-yl)-butyl]-[1,3]dioxane-2-carboxylic acid can be prepared more economically with less process steps under moderate reaction conditions with an outstanding yield. Further, crude intermediate products can often be used in subsequent reaction steps without the need of any additional purification steps. In addition, the process according to the present invention yields high amounts of the desired [cis]-compounds.

The process according to the present invention provides a more efficient method for producing compounds of formula I. Compared to the processes known in the art, the process of the present invention exhibits a higher yield, moderate reaction conditions and other commercially relevant advantages.

Unless otherwise indicated, the following definitions are set forth to illustrate and define the meaning and scope of the various terms used to describe the invention herein.

In this specification the term “lower” is used to mean a group consisting of one to seven, preferably of one to four carbon atoms.

The term “alkyl” refers to a branched or straight chain monovalent saturated aliphatic hydrocarbon radical of one to twenty carbon atoms, preferably one to sixteen carbon atoms.

The term “lower-alkyl” refers to a branched or straight chain monovalent alkyl radical of one to seven carbon atoms, preferably one to four carbon atoms. This term is further exemplified by such radicals as methyl, ethyl, n-propyl, isopropyl, i-butyl, n-butyl, t-butyl and the like with methyl and ethyl being preferred.

The term “fluoro-lower-alkyl” refers to lower-alkyl groups which are mono- or multiply substituted with fluorine. Examples of fluoro-lower-alkyl groups are e.g. CF₃, CF₃CH₂ and (CF₃)₂CH.

The term “alkoxy” refers to the group alkyl-O—, the term “lower alkoxy” to the group lower-alkyl-O—.

The term “fluoro-lower-alkoxy” refers to the group R″—O—, wherein R″ is fluoro-lower-alkyl. Examples of fluoro-lower-alkoxy groups are e.g. CFH₂—O, CF₂H—O, CF₃—O, CF₃CH₂—O, CF₃(CH₂)₂—O, (CF₃)₂CH—O, and CF₂H—CF₂—O.

The term “cycloalkyl” refers to a monovalent carbocyclic radical of 3 to 10 carbon atoms, preferably 3 to 6 carbon atoms, such as e.g. cyclopropyl, cyclobutyl, cyclopentyl or cyclohexyl.

The term “halogen” refers to fluorine, chlorine, and bromine, preferably to chlorine and bromine and more preferably to bromine.

The term “pharmaceutically acceptable salts” refers to salts of compounds of formula (I) with bases. Examples of such salts are alkaline, earth-alkaline and ammonium salts such as e.g. Na—, K—, Ca— and Trimethylammonium salt. Alkaline salts, particularly the Na-salt, are preferred.

In detail, the present invention refers to a process for the preparation of compounds of formula (I)

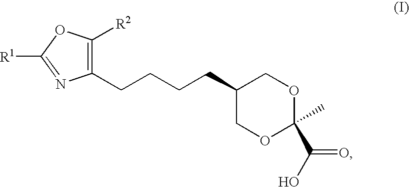

comprising reacting a compound of formula (II)

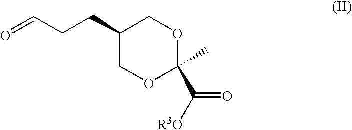

with a compound of formula (III)

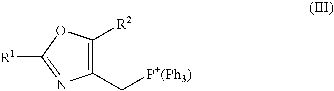

to obtain a compound of formula (IV)

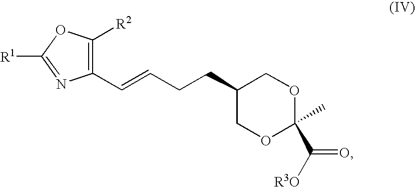

hydrogenating and saponifying the compound of formula (IV) to obtain the compound of formula (I),
wherein
R¹ is phenyl which is optionally substituted with 1-3 substituents independently selected from the group consisting of halogen, lower-alkyl, lower-alkoxy, fluoro-lower-alkyl and fluoro-lower-alkoxy,
R² is hydrogen or lower-alkyl,
R³ is hydrogen, lower-alkyl, cycloalkyl or fluoro-lower-alkyl.

In a preferred process as described above, R¹ is 4-methyl-phenyl. Furthermore, it is also preferred that R² is methyl. In a particularly preferred process as described above, the compound of formula (I) is characterised by formula (Ia)

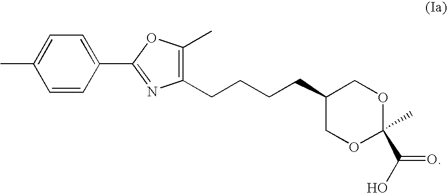

A preferred embodiment of the present invention relates to a process as defined above, wherein the compound of formula (II) is obtained from a compound of formula (IIa)

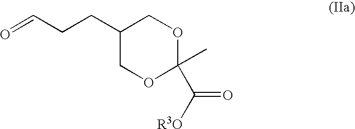

by crystallisation. Preferably, the compound of formula (II) is obtained from the compound of formula (IIa) by crystallisation from an a polar solvent. More preferably, the compound of formula (II) is obtained from the compound of formula (IIa) by crystallisation from diisopropyl ether.

A preferred embodiment of the present invention refers to a process as described above, wherein the compound of formula (II) is obtained by reacting a compound of formula (V)

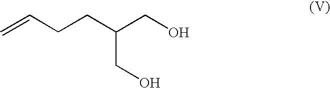

with R³-pyruvate in the presence of BF₃OEt₂ to obtain a compound of formula (VI)

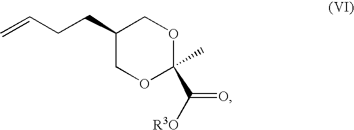

and reacting the compound of formula (VI) with ozone to obtain the compound of formula (II).

Preferably, 0.5-0.9 equivalents of the compound of formula (V), 0.9 to 1.5 equivalents of BF₃OEt₂ and 1 equivalent of R³-pyruvate are used. Preferably, the R³-pyruvate and the BF₃OEt₂ are mixed first and the compound of formula (V) is added subsequently.

Another preferred embodiment of the present invention refers to a process as described above, wherein the compound of formula (II) is obtained by reacting a compound of formula (VII)

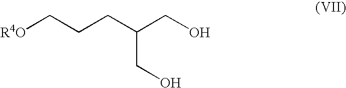

with R³-pyruvate in the presence of BF₃OEt₂ to obtain a compound of formula (VIII)

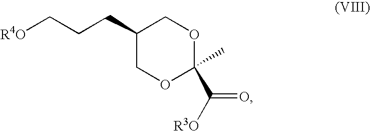

if R⁴ is not H, optionally converting the compound of formula (VIII) to a compound of formula (VIII) in which R⁴ is H, oxidizing the compound of formula (VIII) to obtain the compound of formula (II), wherein R⁴ is hydrogen or benzyl which is optionally substituted with 1 to 3 substituents selected from the group consisting of halogen, lower-alkyl, lower-alkoxy, fluoro-lower-alkyl and fluoro-lower-alkoxy. Preferably, R⁴ is hydrogen, benzyl or 4-methoxy-benzyl.

More preferably, R⁴ is benzyl. Preferably, 0.5-0.9 equivalents of the compound of formula (VII), 0.9 to 1.5 equivalents of BF₃OEt₂ and 1 equivalent of R³-pyruvate are used. Preferably, the R³-pyruvate and the BF₃OEt₂ are mixed first and the compound of formula (VII) is added subsequently. Preferably, R³ is lower-alkyl, more preferably R³ is ethyl.

In the processes for the preparation of a compound of formula (II) as described above, it is preferred that the compound of formula (IIa), i.e. the stereoisomeric mixture, which is produced as an intermediate, is crystallised as described above, in order to obtain the compound of formula (II).

Another embodiment of the present invention refers to a process as defined above for the preparation of [cis]-2-Methyl-5-[4-(5-methyl-2-p-tolyl-oxazol-4-yl)-butyl]-[1,3]dioxane-2-carboxylic acid.

The processes for the preparation of a compound of formula (I) as described above can optionally in addition comprise the conversion of the compound of formula (I) into a pharmaceutically acceptable salt.

Another preferred embodiment of the present invention relates to intermediate compounds and starting compounds of the processes as defined above, particularly to compounds selected from the group consisting of:
[cis]-5-But-3-enyl-2-methyl-[1,3]dioxane-2-carboxylic acid ethyl ester,
[cis]-2-Methyl-5-(3-oxo-propyl)-[1,3]dioxane-2-carboxylic acid ethyl ester,
(5-Methyl-2-p-tolyl-oxazol-4-ylmethyl)-triphenyl-phosphonium chloride,
[cis]-2-Methyl-5-[4-(5-methyl-2-p-tolyl-oxazol-4-yl)-but-3-enyl]-[1,3]acid ethyl ester,
[cis]-5-(3-Benzyloxy-propyl)-2-methyl-[1,3]dioxane-2-carboxylic acid ethyl ester,
[cis]-5-(3-Hydroxy-propyl)-2-methyl-[1,3]dioxane-2-carboxylic acid ethyl ester, and
2-[3-(4-Methoxy-benzyloxy)-propyl]-propane-1,3-diol.

The reaction of compounds of formula (II) with compounds of formula (III) as described above can be carried out according to methods known in the art, e.g. in a solvent such e.g. THF, toluene, DME, CH₂Cl₂, CHCl₃ or Et₂O, or in a mixture of such solvents. The reaction can conveniently be carried out in the presence of reagents such as NaH, KH, BuLi, LDA, LiHMDS, LiPh, t-BuOK, t-BuONa or NEt₃ at a convenient temperature, e.g. in the range of −78° C. up to the boiling temperature of the solvent. It is known to the person skilled in the art that the triphenyl-phosphonium derivatives of formula (III) are positively charged and that they are provided in the form of suitable salts, such as e.g. the Cl, Br, I or mesylate salts, preferably the Cl or Br salt. The reaction mixture therefore also comprises the corresponding counter ions of the compounds of formula (III), such as e.g. Cl⁻, Br⁻, I⁻ or mesylate. The term “Ph”, particularly in formula (III), refers to a phenyl group. The ratio of the compound of formula (II) to the compound of formula (III) can vary, e.g. in the range of 1/2 to 2/1.

The hydrogenation of a compound of formula (IV) can be carried out by methods known to the person skilled in the art, e.g. by transfer hydrogenation or applying various Pd, Pt, Rh, Re catalysts like Pd/C, PtO₂, Zn or Raney Nickel, in a solvent such e.g. THF, toluene, EtOH, MeOH, EtOAc or AcOH, or in a mixture of such solvents at a convenient temperature, e.g. in the range of −20° C. up to the boiling temperature of the solvent.

The saponification of a compound of formula (IV), or of a hydrogenated compound of formula (IV) respectively, can be carried out by methods known to the person skilled in the art, e.g. under basic conditions, in the presence of NaOH, KOH, LiOH, K₂CO₃, Na₂CO₃, NaHCO₃ or Ba(OH)₂, or under acidic conditions, e.g. in the presence of H₂SO₄ or HCl, in a solvent such e.g. THF, toluene, EtOH, MeOH, EtOAc, acetone, dioxane or water, or in a mixture of such solvents, at a convenient temperature, e.g. in the range of −20° C. up to the boiling temperature of the solvent.

The order, in which the hydrogenation and saponification are carried out can be exchanged, if desired.

The compounds of formula (II) can be obtained from compounds of formula (IIa) by crystallisation according to methods known to the person skilled in the art, e.g. by dissolving the compound in an appropriate solvent like diisopropyl ether under heating, treating the solution with an additional solvent like hexane or heptane and cooling after seeding or without seeding the solution or suspension to e.g. −20° C. The resulting suspension can be stirred at −20° C. and filtered. The crystals can be rinsed with e.g. cold diisopropyl ether and dried.

The reaction of compounds of formula (V) with R³-pyruvate as described above can conveniently be carried out according to methods known to the person skilled in the art, e.g. in a suitable solvent such as CH₂Cl₂, pentane, cyclohexane, toluene, diethyl ether or mixtures thereof, or in the absence of solvents. In order to obtain good cis/trans ratios, it is preferred that the reaction occurs fast, which can be achieved by selecting an appropriate order and appropriate ratios, in which the reactants are mixed as described before. The reaction can be carried out at a suitable temperature, e.g. in the range between −20° C. and the boiling point of the solvent, preferably between room temperature and 50° C.

The compound R³-pyruvate can be characterised by formula (IX)

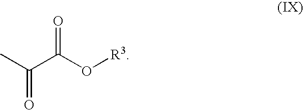

The reaction of a compound of formula (VI) with ozone as described above can conveniently be carried out according to methods known to the person skilled in the art. The reaction can be carried out in a solvent such as e.g. CH₂Cl₂, CHCl₃, EtOAc, AcOH, DMF or MeOH, or mixtures thereof, at suitable temperatures, e.g. in the range between −65° C. and −20° C. The transformation can also conveniently be carried out e.g. with NaIO₄ and OSO₄, AD-mix or RuCl₃, or MCPBA and HIO₄ in a solvent such as e.g. CH₂Cl₂, CHCl₃, THF, water, dioxane or CH₃CN or mixtures thereof, at suitable temperatures, e.g. in the range between −20° C. and the boiling point of the solvent.

The reaction of compounds of formula (VII) with R³-pyruvate as described above can conveniently be carried out according to methods known to the person skilled in the art, e.g. in a suitable solvent such as CH₂Cl₂, pentane, cyclohexane, toluene, diethyl ether or mixtures thereof, or in the absence of solvents. In order to obtain good cis/trans ratios, it is preferred that the reaction occurs fast, which can be achieved by selecting an appropriate order and appropriate ratios, in which the reactants are mixed as described before. The reaction can be carried out at a suitable temperature, e.g. in the range between −20° C. and the boiling point of the solvent, preferably between room temperature and 50° C.

The compound of formula (VIII), in which R⁴ is not H, can be converted to a compound of formula (VIII) in which R⁴ is H, by methods known to the person skilled in the art, e.g. by dissolving the compound in a solvent such as e.g. MeOH, EtOH, dioxane, AcOH, cyclohexane, CH₂Cl₂, CHCl₃ or EtOAc or mixtures thereof, and treatment with Pd, Pd(OAc)₂ on carbon, Pd (OH)₂, Raney nickel, BF₃OEt₂, BCl₃ or with DDQ, ceric ammonium nitrate or NBS at suitable temperatures, e.g. in the range between −20° C. and the boiling temperature of the solvent.

Compounds of formula (VIII) can be oxidized to obtain compounds of formula (II) by methods known to the person skilled in the art, e.g. by dissolving the compound in a solvent such as e.g. CH₂Cl₂, CH₃CN, toluene, water, Et₂O, cyclohexane or DMSO and treatment under Swern conditions, with TEMPO and bleach, CrO₃, pyridinium chlorochromate, P₂O₅, Mn(OAc)₂ or MnO₂ at suitable temperatures, e.g. in the range between −20° C. and the boiling temperature of the solvent.

The conversion of a compound of formula (I) into a pharmaceutically acceptable salt can be carried out by methods known in the art, e.g. under basic conditions with NaOH, KOH, LiOH, K₂CO₃, Na₂CO₃, NaHCO₃ or Ba(OH)₂ in a solvent such e.g. THF, toluene, EtOH, MeOH, EtOAc, acetone, dioxane or water or in a mixture of such solvents at a convenient temperature, e.g. in the range of −20° C. up to the boiling temperature of the solvent.

The following examples shall illustrate preferred embodiments of the present invention but are not intended to limit the scope of the invention. Starting materials are commercially available, known in the art, or can be prepared according to procedures well known to the person skilled in the art or in analogy thereto.

### EXAMPLES

### Example 1

### 2-But-3-enyl-malonic acid diethyl ester

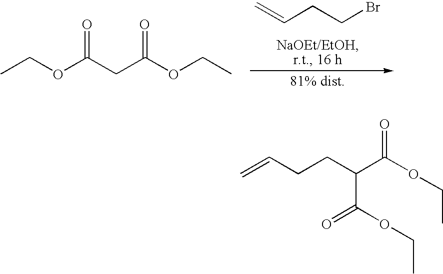

A solution of 50.9 g (157.1 mmol) sodium ethylate (21% in EtOH) in 100 ml EtOH was treated at room temperature with 25.9 g (157.1 mmol) diethyl malonate. The reaction mixture was heated to 50° C., stirred for 30 min at this temperature, cooled to room temperature and treated within 1 h with 15.2 g (110 mmol) 4-bromo-1-butene. The reaction mixture was stirred over night, the solvent evaporated and the residue treated with 100 ml pentane and 20 ml water. After extraction, the phases were separated and the organic phase was washed with saturated aqueous sodium chloride. The aqueous phases were extracted with pentane. The combined organic extract was dried, concentrated in a rotary evaporator and dried under high vacuum at room temperature to provide 27.5 g crude product. Distillation at 80° C. to 100° C. and 0.07 mbar yielded 19.1 g (81%) 2-but-3-enyl-malonic acid diethyl ester as a colorless oil.

MS (El): m/e 214 (M, 1), 160 (100), 133 (58), 123 (70), 114 (22), 105 (18), 95 (49), 86 (36), 67 (29).

### Example 2

### 2-But-3-enyl-propane-1,3-diol

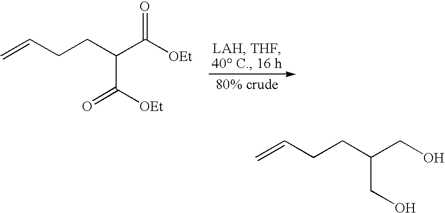

A suspension of 9.0 g (230.0 mmol) lithium aluminum hydride in 140 ml THF was treated at 20-30° C. within 1 h with a solution of 33.5 g (156.2 mmol) 2-but-3-enyl-malonic acid diethyl ester in 60 ml THF (exothermic reaction). The white suspension was stirred at room temperature for 1 h, heated to 45-50° C. and stirred over night. The mixture was cooled to room temperature, slowly treated with 15 ml water (strong gas formation), 7.5 ml sodium hydroxide and again with 30 ml water. The white suspension (pH 10) was stirred for 30 min and than adjusted the pH to 7 with ca. 25 ml aq. HCl (25%). The suspension was filtered over hyflo and the filter cake rinsed with 50 ml TMBE and three times with 20 ml CH₂Cl₂. The solvent of the filtrate was evaporated to get 22.1 g (quant.) crude 2-but-3-enyl-propane-1,3-diol as a colorless oil.

MS (ISP): m/e=153 (M+Na⁺, 78). 148 (M+NH₄⁺, 54), 131 (M+H⁺, 100), 113 (43).

### Example 3

### cis]-5-But-3-enyl-2-methyl-[1,3]dioxane-2-carboxylic acid ethyl ester

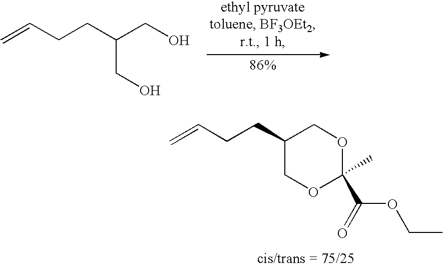

A solution of 5.7 ml (49.8 mmol) ethyl pyruvate, 5.7 ml (45.0 mmol) boron trifluoride-diethyl etherate in 45 ml toluene was treated within 40 min dropwise with a solution of 4.5 g (34.4 mmol) 2-but-3-enyl-propane-1,3-diol in 25 ml toluene and stirred at room temperature for 1 h. The reaction mixture was treated with 60 ml water and 50 ml EtOAc. After extraction, the phases were separated and the organic phase was washed with half saturated aqueous sodium chloride. The aqueous phases were extracted with EtOAc. The combined organic extract was dried, concentrated in a rotary evaporator and dried under high vacuum at room temperature to provide after chromatographic purification 6.7 g (86%) colorless oil of 5-but-3-enyl-2-methyl-[1,3]dioxane-2-carboxylic acid ethyl ester as a cis/trans=75/25 mixture.

MS (ISP): m/e=251 (M+Na⁺, 100), 229 (M+H⁺, 100), 215 (15), 183 (26), 155 (71).

### Example 4

### [cis]-2-Methyl-5-(3-oxo-propyl)-[1,3]dioxane-2-carboxylic acid ethyl ester

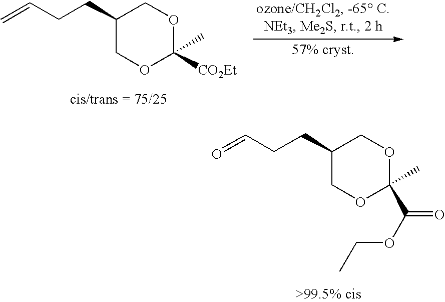

A solution of 5.4 g (23.6 mmol) 5-but-3-enyl-2-methyl-[1,3]dioxane-2-carboxyl acid ethyl ester as a cis/trans=75/25 mixture in 75 ml CH₂Cl₂ was treated at −65° C. with ozone (the course of the reaction was monitored by TLC). After 30 min the reaction mixture was treated at −10-0° C. with 12.0 g (118.2 mmol) triethly amine and 7.4 g (118.2 mmol) dimethyl sulfide. The reaction mixture was warmed to room temperature, stirred for 2 h at this temperature and treated with 25 ml CH₂Cl₂ and 25 ml saturated aqueous sodium chloride. After extraction, the phases were separated and the organic phase was washed twice with 25 ml citric acid and with 20 ml saturated aqueous sodium chloride. The aqueous phases were extracted with CH₂Cl₂. The combined organic extract was dried, concentrated in a rotary evaporator and dried under high vacuum at room temperature to yield 4.8 g (88%) crude product cis/trans=77/23. Crystallization from diisopropyl ether at −20° C. gave 3.1 g (57%) white crystals of 2-methyl-5-(3-oxo-propyl)-[1,3]dioxane-2-carboxylic acid ethyl ester as a mixture cis/trans=96/4. A second crystallization from diisopropyl ether at −20° C. gave 2-methyl-5-(3-oxo-propyl)-[1,3]dioxane-2-carboxylic acid ethyl ester as a mixture cis/trans>99.5/0.5. M.p.=38.5-39° C. x-Ray Nr. 1421.

MS (ISP): m/e=253 (M+Na⁺, 100), 248 (M+NH₄⁺, 49), 231 (M+H⁺, 34), 213 (13), 171 (31).

### Example 5

### 4,5-Dimethyl-2-p-tolyl-oxazole 3-oxide

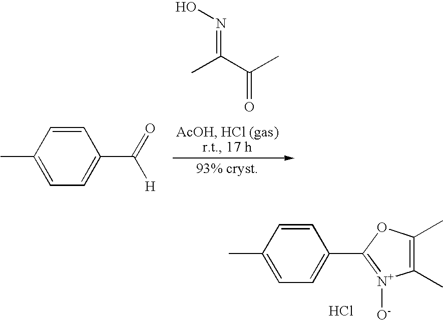

A solution of 10.0 g (81.6 mmol) tolualdehyde and 8.4 g (82.3 mmol) 2,3-butadion-monoxime in 100 ml acetic acid was treated at 5° C. for 2 h with HCl gas and stirred for 17 h at room temperature. The reaction mixture was diluted with 100 ml CH₂Cl₂ (gas evolution) and subsequent CH₂Cl₂ and ca. 50 ml acetic acid evaporated. 200 ml TBME was added to the residual yellow solution, whereby a suspension was formed. After stirring for 30 min at room temperature the suspension was filtrated, the crystals washed with TBME and dried to get 18.2 g (93%) 4,5-dimethyl-2-p-tolyl-oxazole 3-oxide as white powder. M.p.=182-184° C.

MS (ISP): m/e=204 (M+H⁺, 100), 187 (10).

### Example 6

### 4-Chloromethyl-5-methyl-2-p-tolyl-oxazole

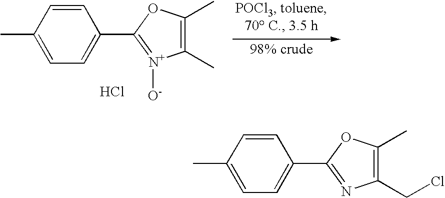

A white suspension of 6.2 g (25.7 mmol) 4,5-dimethyl-2-p-tolyl-oxazole 3-oxide in 60 ml toluene was treated with 2.6 ml (27.8 mmol) phosphorous oxychloride, heated to 70° C. and stirred for 3.5 h at this temperature. The reaction mixture was poured onto a mixture of 100 ml 2N aqueous NaOH and 100 ml ice and stirred for 5 min (pH=10). After extraction the phases were separated, the aqueous phase was extracted twice with 100 ml EtOAc and the organic phases were washed with saturated aqueous sodium chloride. The combined organic extract was dried and concentrated under reduced pressure to yield 5.6 g (98%) 4-chloromethyl-5-methyl-2-p-tolyl-oxazole as beige powder. M.p.=91.5-92° C.

MS (ISP): m/e=224 (M+H⁺, 40), 222 (M+H⁺, 100), 186 (32).

### Example 7

### (5-Methyl-2-p-tolyl-oxazol-4-ylmethyl)-triphenyl-phosphonium chloride

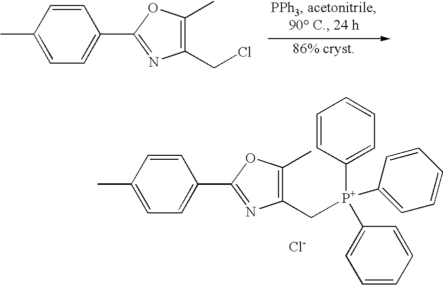

A solution of 1.0 g (4.3 mmol) 4-chloromethyl-5-methyl-2-p-tolyl-oxazole and 1.2 g (4.3 mmol) triphenyl phosphine in acetonitrile were heated to 90° C. and stirred for 24 h at this temperature. The reaction mixture was cooled to 0° C. and the so formed suspension filtrated. The crystals were washed twice with 10 ml acetonitrile, dried at room temperature under high vacuum to yield 1.8 g (86%) (5-methyl-2-p-tolyl-oxazol-4-ylmethyl)-triphenyl-phosphonium chloride. M.p.=135-138° C.

MS (ISP): m/e=448 (M+H⁺, 100).

### Example 8

### [cis]-2-Methyl-5-[4-(5-methyl-2-p-tolyl-oxazol-4-yl)-butyl]-[1,3]dioxane-2-carboxylic acid ethyl ester

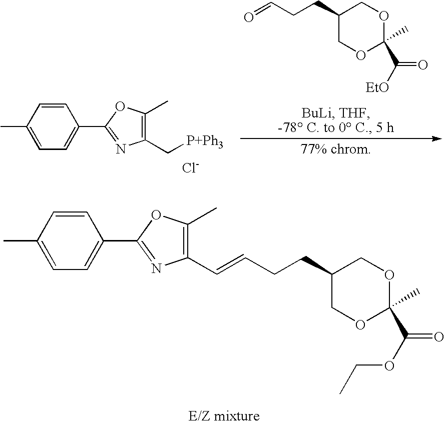

A white suspension of 2.3 g (4.8 mmol) (5-methyl-2-p-tolyl-oxazol-4-ylmethyl)-triphenyl-phosphonium chloride in 100 ml THF was treated within 5 min at −70° C. with 3.0 ml (4.8 mmol) butyl lithium. The orange suspension was stirred at −70° C. for 1 h and treated within 30 min with a solution of 1 g (4.3 mmol) [cis]-2-methyl-5-(3-oxo-propyl)-[1,3]dioxane-2-carboxylic acid ethyl ester in 25 ml THF. The reaction mixture was stirred at −70° C. for 1 h and slowly warmed to room temperature. The reaction mixture was diluted with 50 ml EtOAc and 20 ml saturated aq. NH₄Cl solution. After extraction, the phases were separated and the organic phase was washed with saturated aqueous NH₄Cl solution. The aqueous phases were extracted twice with 10 ml EtOAc. The combined organic extract was dried, concentrated in a rotary evaporator and dried under high vacuum at room temperature to provide after chromatographic purification 1.3 g (77%) yellow oil of [cis]-2-methyl-5-[4-(5-methyl-2-p-tolyl-oxazol-4-yl)-but-3-enyl]-[1,3]dioxane-2-carboxylic acid ethyl ester as an E/Z-mixture. Alternatively to the chromatographic purification an extraction with heptane and methanol/water 8/2 yielded also the desired product in 88% yield

MS (ISP): m/e=422 (M+Na⁺, 11), 400 (M+H⁺, 100), 284 (19).

### Example 9

### [cis]-2-Methyl-5-[4-(5-methyl-2-p-tolyl-oxazol-4-yl)-butyl]-[1,3]dioxane-2-carboxylic acid ethyl ester

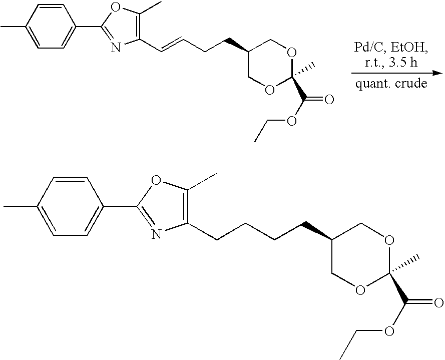

A solution of 320 mg (0.8 mmol) [cis]-2-Methyl-5-[4-(5-methyl-2-p-tolyl-oxazol-4-yl)-but-3-enyl]-[1,3]dioxane-2-carboxylic acid ethyl ester as an E/Z-mixture in 5 ml EtOH was treated with 40 mg Pd/C 5% E101 N/D and hydrogen. The black suspension was stirred at room temperature under ca. 1 bar hydrogen pressure for 3.5 h. The suspension was filtrated and the residue rinsed with 5 ml EtOH, the solvent of the filtrate was evaporated under reduced pressure and the residue dried under high vacuum at room temperature to get 306 mg (96%) yellow oil of crude [cis]-2-Methyl-5-[4-(5-methyl-2-p-tolyl-oxazol-4-yl)-butyl]-[1,3]dioxane-2-carboxylic acid ethyl ester.

### MS (EI): m/e=401 (M, 10), 328 (100), 43 (19).

### Example 10

### [cis]-2-Methyl-5-[4-(5-methyl-2-p-tolyl-oxazol-4-yl)-butyl]-[1,3]dioxane-2-carboxylic acid

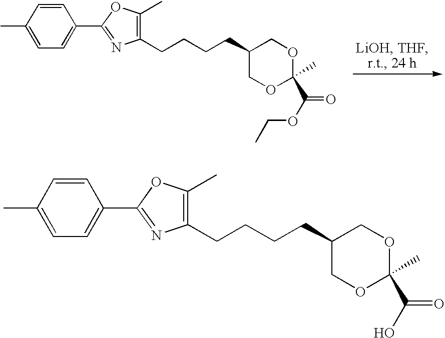

A solution of 200 mg (0.5 mmol) [cis]-2-Methyl-5-[4-(5-methyl-2-p-tolyl-oxazol-4-yl)-butyl]-[1,3]dioxane-2-carboxylic acid ethyl ester in 5 ml THF was treated at room temperature with a solution of 24.3 mg (1.0 mmol) LiOH and 2 ml water. The reaction mixture was stirred at room temperature for 24 h and then treated with 2 ml water and 2 ml diethyl ether. After extraction, the phases were separated and the organic phase was washed with 2 ml water. The pH of the aqueous phase was adjusted from ca. 14 to 1 with aq. HCl and extracted twice with 10 ml EtOAc. The organic phases were washed twice with 2 ml saturated aqueous sodium chloride, dried and evaporated under reduced pressure. The residue was dried under high vacuum at room temperature to get 177.5 mg (95%) [cis]-2-methyl-5-[4-(5-methyl-2-p-tolyl-oxazol-4-yl)-butyl]-[1,3]dioxane-2-carboxylic acid as white crystals. M.p.=149-150° C.

MS (ISN): m/e=372 (M−H⁺, 100).

### Example 11

### 2-(3-Benzyloxy-propyl)-malonic acid diethyl ester

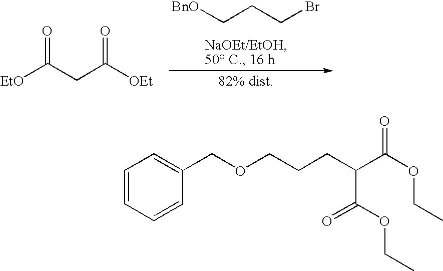

A brown solution of 100.0 ml (271.6 mmol, 21% in EtOH) sodium ethylate in 450 ml EtOH was treated within 40 min at room temperature with a solution of 47.0 g (290.5 mmol) diethyl malonate in 90 ml EtOH. The reaction mixture was heated to 30° C., stirred for 2.5 h at this temperature and treated within 15 min with 45.0 g (192.5 mmol) benzyl-3-bromopropyl ether. The reaction mixture was heated to 50° C. and stirred over night, the solvent was evaporated and the residue treated with 500 ml EtOAc and 300 ml half saturated aqueous sodium chloride. After extraction, the phases were separated and the organic phase was washed with saturated aqueous sodium chloride. The aqueous phases were extracted twice with EtOAc. The combined organic extract was dried, concentrated in a rotary evaporator and dried under high vacuum at room temperature to provide 71.1 g crude product. Distillation at 180° C. to 195° C. and 0.07 mbar yielded 48.9 g (82%) 2-(3-benzyloxy-propyl)-malonic acid diethyl ester as a colorless oil.

MS (ISP): m/e=309 (M+H⁺, 10), 263 (4), 217 (13), 201 (12), 173 (68), 127 (19), 91 (100).

### Example 12

### 2-(3-Benzyloxy-propyl)-propane-1,3-diol

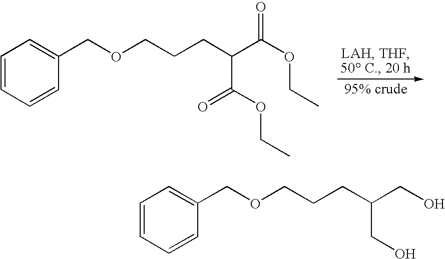

A suspension of 1.8 g lithium aluminum hydride in 70 ml THF was treated dropwise within 15 min at room temperature with a solution of 7.0 g (22.7 mmol) 2-(3-benzyloxy-propyl)-malonic acid diethyl ester in 7 ml THF (exothermic reaction). The grey suspension was stirred for 20 h at 50° C., cooled to room temperature and treated dropwise with 50 ml water (exothermic reaction, gas evolution). 50 ml EtOAc was added to the white suspension. The suspension was filtered over dicalite and the filtrate evaporated under reduced pressure and dried under high vacuum at room temperature to yield 4.8 g (95%) 2-(3-benzyloxy-propyl)-propane-1,3-diol as a colorless oil.

MS (ISP): m/e=247 (M+Na⁺, 52), 225 (M+H⁺, 100).

### Example 13

### [cis]-5-(3-Benzyloxy-propyl)-2-methyl-[1,3]dioxane-2-carboxylic acid ethyl ester

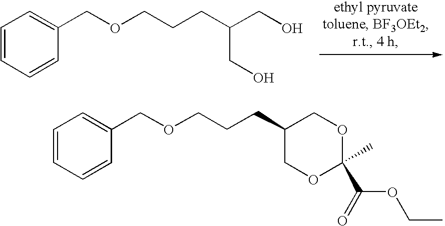

A colorless solution of 11.5 ml (100.4 mmol) ethyl pyruvate in 12.5 ml (98.6 mmol) boron trifluoride-diethyl etherate in 170 ml CH₂Cl₂ was treated at room temperature within 40 min dropwise with a solution of 14.8 g (65.9 mmol) 2-(3-benzyloxy-propyl)-propane-1,3-diol in 30 ml toluene and stirred at room temperature for 4 h. The reaction mixture was treated with 150 ml half saturated aqueous sodium chloride. After extraction, the phases were separated and the organic phase was washed with half saturated aqueous sodium chloride. The aqueous phases were extracted twice with CH₂Cl₂. The combined organic extracts was dried, concentrated in a rotary evaporator and dried under high vacuum at room temperature to provide after filtration over silica gel 19.6 g (93%) colorless oil of 5-(3-benzyloxy-propyl)-2-methyl-[1,3]dioxane-2-carboxylic acid ethyl ester as a cis/trans=75/25 mixture.

MS (ISP): m/e=323 (M+H⁺, 2), 249 (14), 91 (100).

### Example 14

### [cis]-5-(3-Hydroxy-propyl)-2-methyl-[1,3]dioxane-2-carboxylic acid ethyl ester

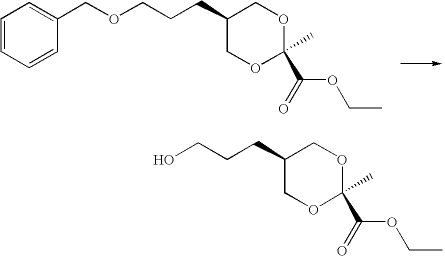

A colorless solution of 19.4 g (60.0 mmol) 5-(3-benzyloxy-propyl)-2-methyl-[1,3]dioxane-2-carboxylic acid ethyl ester as a cis/trans=75/25 mixture in 200 ml EtOH was treated at room temperature with 2 g Pd/C 10% (Degussa) and hydrogen. The black suspension was stirred at room temperature under ca. 1 bar hydrogen pressure for 1 h. The suspension was filtrated and the residue rinsed with 25 ml EtOH, the solvent of the filtrate was evaporated under reduced pressure and the residue dried under high vacuum at room temperature to get 13.3 g (95%) colorless oil of 5-(3-hydroxy-propyl)-2-methyl-[1,3]dioxane-2-carboxylic acid ethyl ester as a cis/trans=75/25 mixture.

MS (ISP): m/e=255 (M+Na⁺, 78), 250 (M+NH₄⁺, 28), 233 (M+H⁺, 100).

### Example 15

### [cis]-2-Methyl-5-(3-oxo-propyl)-[1,3]dioxane-2-carboxylic acid ethyl ester

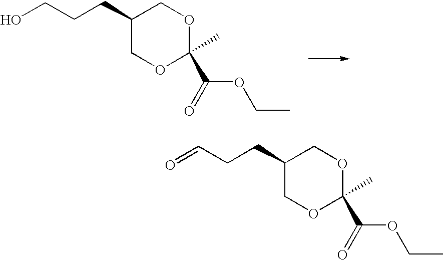

A colorless solution of 10.0 g (43.1 mmol) 5-(3-hydroxy-propyl)-2-methyl-[1,3]dioxane-2-carboxylic acid ethyl ester as a cis/trans=75/25 mixture in 100 ml CH₂Cl₂ was treated at room temperature with 1 g (6.3 mmol) TEMPO. To the orange solution was added 615.0 mg (5.2 mmol) KBr and 30 ml 10%-aqueous NaHCO₃. The two-phasic mixture was treated at room temperature within 45 min with 60 ml (100.8 mmol) sodium hypochlorite (exothermic reaction). The reaction was stirred for 30 min at room temperature and treated with 100 ml 10%-aqueous sodium chloride and 25 ml CH₂Cl₂. After extraction, the phases were separated and the organic phase was washed with half saturated aqueous Na₂CO₃ and with half saturated aqueous sodium chloride. The aqueous phases were extracted twice with CH₂Cl₂. The combined organic extract was dried, concentrated in a rotary evaporator and dried under high vacuum at room temperature to provide after crystallization from diisopropyl ether 5.6 g (57%) white crystals of [cis]-2-methyl-5-(3-oxo-propyl)-[1,3]dioxane-2-carboxylic acid ethyl ester.

MS (ISP): m/e=253 (M+Na⁺, 100), 248 (M+NH₄⁺, 47), 231 (M+H⁺, 31), 213 (8).

### Example 16

### 2-[3-(4-Methoxy-benzyloxy)-propyl]-malonic acid diethyl ester

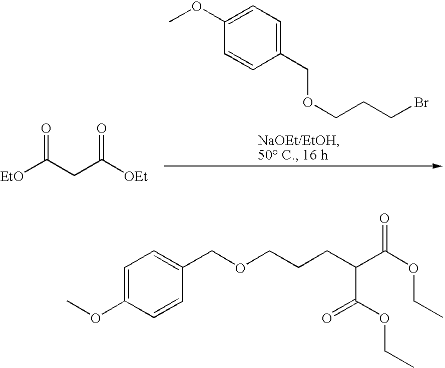

A solution of 7.0 g (21.6 mmol, 21% in EtOH) sodium ethylate in 50 ml EtOH was treated within 30 min at room temperature with a solution of 3.8 g (23.2 mmol) diethyl malonate in 10 ml EtOH. The reaction mixture was heated to 30° C., stirred for 4 h at this temperature and treated within 15 min with 4.0 g (15.4 mmol) 4-methoxy-benzyl-3-bromopropyl ether. The reaction mixture was heated to 50° C. and stirred over night, the solvent was evaporated and the residue treated with 50 ml EtOAc and 10 ml saturated aqueous sodium chloride. After extraction, the phases were separated and the organic phase was washed twice with saturated aqueous sodium chloride. The aqueous phases were extracted twice with 15 ml EtOAc. The combined organic extract was dried, concentrated in a rotary evaporator and dried under high vacuum at room temperature to yield after chromatographic purification 3.9 g (75%) 2-[3-(4-methoxy-benzyloxy)-propyl]-malonic acid diethyl ester as a colorless oil.

MS (EI): m/e=338 (M, 7), 217 (4), 202 (7), 217 (13), 173 (38), 121 (100).

### Example 17

### 2-[3-(4-Methoxy-benzyloxy)-propyl]-propane-1,3-diol

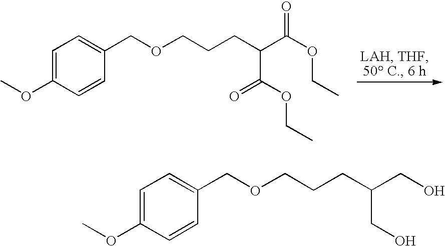

A suspension of 71.1 mg (1.8 mmol) lithium aluminum hydride in 5 ml THF was treated dropwise within 5 min at room temperature with a solution of 300.0 mg (0.9 mmol) 2-[3-(4-methoxy-benzyloxy)-propyl]-malonic acid diethyl ester in 3 ml THF (exothermic reaction). The grey suspension was stirred for 5.5 h at 50° C., cooled to room temperature and treated dropwise with 2 ml water (exothermic reaction, gas evolution). 5 ml THF was added to the white suspension. The suspension was filtered over dicalite, rinsed with 10 ml CH₂Cl₂. After extraction, the phases were separated and the organic phase was washed twice with half saturated aqueous sodium chloride. The aqueous phases were extracted twice with 10 ml CH₂Cl₂. The combined organic extract was dried, concentrated in a rotary evaporator under reduced pressure and dried under high vacuum at room temperature to yield 118.0 mg (52%) 2-[3-(4-methoxy-benzyloxy)-propyl]-propane-1,3-diol as a colorless oil.

MS (EI): m/e=254 (M, 4), 137 (100), 121 (84), 109 (5), 77 (6).

### Example 18

### [cis]-5-(3-Hydroxy-propyl)-2-methyl-[1,3]dioxane-2-carboxylic acid ethyl ester

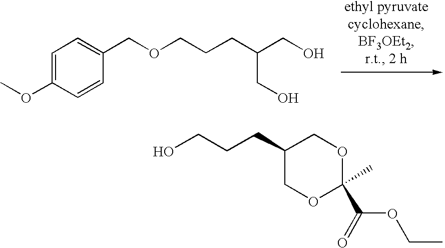

A colorless solution of 270 μl (2.4 mmol) ethyl pyruvate and 324 μl (2.6 mmol) boron trifluoride-diethyl etherate in 5 ml cyclohexane was treated at room temperature within 10 min dropwise with a solution of 0.5 g (2.0 mmol) 2-[3-(4-methoxy-benzyloxy)-propyl]-propane-1,3-diol in 1 ml CH₂Cl₂ and stirred at room temperature for 2 h. The reaction mixture was treated with 10 ml CH₂Cl₂ and 3 ml water. After extraction, the phases were separated and the water phase was extracted twice with 3 ml CH₂Cl₂. The combined organic extract was dried, concentrated in a rotary evaporator and dried under high vacuum at room temperature to provide after filtration over silica gel 192.0 mg (42%) colorless oil of 5-(3-hydroxy-propyl)-2-methyl-[1,3]dioxane-2-carboxylic acid ethyl ester as a cis/trans=ca. 75/25 mixture.

MS (ISP): m/e=255 (M+Na⁺, 76), 250 (M+NH₄⁺, 26), 233 (M+H⁺, 100), 159 (9).

### Example 19

### [cis]-5-(3-Hydroxy-propyl)-2-methyl-[1,3]dioxane-2-carboxylic acid ethyl ester

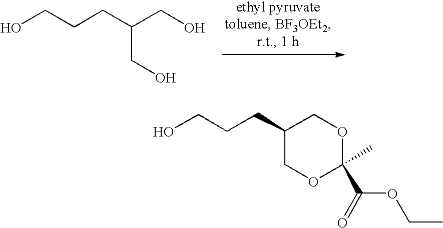

A colorless solution of 111 μl (0.97 mmol) ethyl pyruvate and 132 μl (1.04 mmol) boron trifluoride-diethyl etherate in 1 ml toluene was treated at room temperature within 1 min dropwise with a 30° C. warm solution of 100 mg (0.75 mmol) 2-hydroxymethyl-pentane-1,5-diol in 7 ml toluene and stirred at room temperature for 1 h. The reaction mixture was treated with 5 ml CH₂Cl₂ and 1 ml water. After extraction, the phases were separated and the organic phase was washed twice with saturated aqueous sodium chloride. The water phase was extracted twice with 2 ml CH₂Cl₂. The combined organic extract was dried, concentrated in a rotary evaporator and dried under high vacuum at room temperature to provide after filtration over silica gel 5-(3-hydroxy-propyl)-2-methyl-[1,3]dioxane-2-carboxylic acid ethyl ester as a cis/trans=ca. 75/25 mixture in 52% yield.

MS (ISP): m/e=255 (M+Na⁺, 100), 250 (M+NH₄⁺, 27), 233 (M+H⁺, 95), 159 (10).

It is to be understood that the invention is not limited to the particular embodiments of the invention described above, as variations of the particular embodiments may be made and still fall within the scope of the appended claims.

What is claimed is:

1. A process for the preparation of a compound of formula (I) 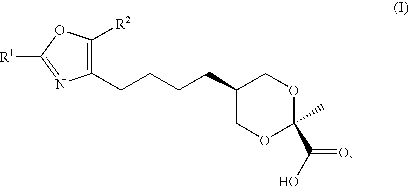
comprising the steps of reacting a compound of formula (II) 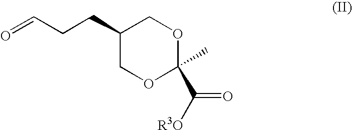
with a compound of formula (III) 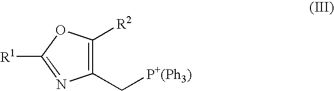
to obtain a compound of formula (IV) 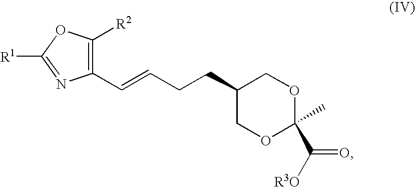
and hydrogenating and saponifying the compound of formula (IV) to obtain the compound of formula (I),
wherein
R¹ is phenyl which is optionally substituted with 1-3 substituents independently selected from the group consisting of halogen, lower-alkyl, lower-alkoxy, fluoro-lower-alkyl and fluoro-lower-alkoxy,
R² is hydrogen or lower-alkyl, and
R³ is hydrogen, lower-alkyl, cycloalkyl or fluoro-lower-alkyl.

2. The process according to claim 1, wherein R¹ is 4-methyl-phenyl.

3. The process according to claim 1, wherein R² is methyl.

4. The process according to claim 1, wherein the compound of formula (I) is characterised by formula (Ia)

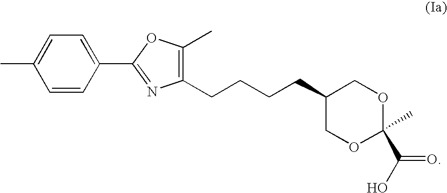

5. The process according to claim 1, wherein the compound of formula (II) is obtained from a compound of formula (IIa) 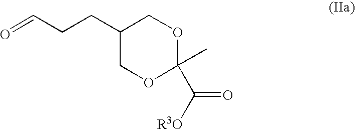
by crystallisation.

6. The process according to claim 5, wherein the compound of formula (II) is obtained from the compound of formula (IIa) by crystallisation from an a polar solvent.

7. The process according to claim 1, wherein the compound of formula (II) is obtained from the compound of formula (IIa) by crystallisation from diisopropyl ether.

8. The process according to claim 1, wherein the compound of formula (II) is obtained by reacting a compound of formula (V) 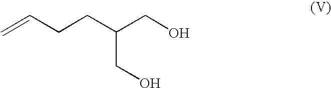
with R³-pyruvate in the presence of BF₃OEt₂ to obtain a compound of formula (VI) 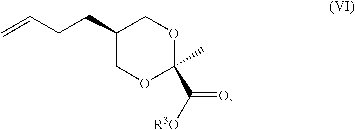
and reacting the compound of formula (VI) with ozone to obtain the compound of formula (II).

9. The process according to claim 8, wherein 0.5-0.9 equivalents of the compound of formula (V), 0.9 to 1.5 equivalents of BF₃OEt₂ and 1 equivalent of R³-pyruvate are used.

10. The process according to claim 1, wherein the R³-pyruvate and the BF₃OEt₂ are mixed first and the compound of formula (VII) is added subsequently.

11. The process according to claim 1, wherein the compound of formula (II) is obtained by reacting a compound of formula (VII) 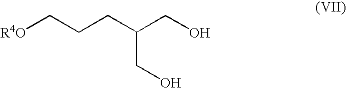
with R³-pyruvate in the presence of BF₃OEt₂ to obtain a compound of formula (VIII) 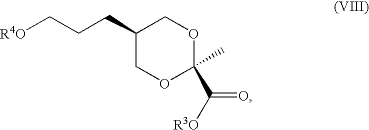
if R⁴ is not H, optionally converting the compound of formula (VIII) to a compound of formula (VIII) in which R⁴ is H,
oxidizing the compound of formula (VIII) to obtain the compound of formula (II),
wherein R⁴ is hydrogen or benzyl which is optionally substituted with 1 to 3 substituents selected from the group consisting of halogen, lower-alkyl, lower-alkoxy, fluoro-lower-alkyl and fluoro-lower-alkoxy.

12. The process according to claim 11, wherein R⁴ is hydrogen, benzyl or 4-methoxy-benzyl.

13. The process according to claim 12, wherein R⁴ is benzyl.

14. The process according to claim 1, wherein 0.5-0.9 equivalents of the compound of formula (VII), 0.9 to 1.5 equivalents of BF₃OEt₂ and 1 equivalent of R³-pyruvate are used.

15. The process according to claim 1, wherein the R³-pyruvate and the BF₃OEt₂ are mixed first and the compound of formula (VII) is added subsequently.

16. The process according to claim 1, wherein R³ is lower-alkyl.

17. The process according to claim 16, wherein R³ is ethyl.

18. A compound selected from the group consisting of:
[cis]-5-But-3-enyl-2-methyl-[1,3]dioxane-2-carboxylic acid ethyl ester,
[cis]-2-Methyl-5-(3-oxo-propyl)-[1,3]dioxane-2-carboxylic acid ethyl ester,
(5-Methyl-2-p-tolyl-oxazol-4-ylmethyl)-triphenyl-phosphonium chloride,
[cis]-2-Methyl-5-[4-(5-methyl-2-p-tolyl-oxazol-4-yl)-but-3-enyl]-[1,3]acid ethyl ester,
[cis]-5-(3-Benzyloxy-propyl)-2-methyl-[1,3]dioxane-2-carboxylic acid ethyl ester,
[cis]-5-(3-Hydroxy-propyl)-2-methyl-[1,3]dioxane-2-carboxylic acid ethyl ester, and
2-[3-(4-Methoxy-benzyloxy)-propyl]-propane-1,3-diol.
